(19) United States
(12) Plant Patent Application Publication
(10) Pub. No.: US 2006/0021099 P1
(43) Pub. Date: Jan. 26, 2006

(54) Pear tree named 'Nellie'

(50) PYRUS COMMUNIS; PEAR

(76) Inventor: Allan G. White, Havelock North (NZ)

Correspondence Address: GREENLEE WINNER AND SULLIVAN P C, 4875 PEARL EAST CIRCLE, SUITE 200, BOULDER, CO, 80301, US

(21) Appl. No.: 10/899,887

(22) Filed: Jul. 26, 2004

(51) Int. Cl. A01H 5/00

(52) U.S. Cl. PLT/176

(57) ABSTRACT

A new distinct pear variety is described. This variety results from crossing the pear varieties ‘Packham's Triumph’ (not patented) and ‘President Heron’ (not patented). The new variety has been named ‘Nellie’ and was selected because of the superior eating quality of the fruit. The fruit of this new variety is characterised by its short pyriform shape, russeted finish and good storage ability.

### GENUS AND SPECIES OF PLANT CLAIMED

Pyrus communis

### BACKGROUND TO THE INVENTION

A new distinct pear variety is described. This variety results from crossing the pear varieties ‘Packham's Triumph’ (not patented) and ‘President Heron’ (not patented). The new variety has been named ‘Nellie’ and was selected because of the superior eating quality of the fruit. The fruit of this new variety is characterised by its short pyriform shape, russeted finish and good storage ability.

### SUMMARY OF THE INVENTION

This new variety was selected from a population of seedlings derived from crossing the pear varieties ‘Packham's Triumph’ and ‘President Heron’. The cross was made in 1983 at Hawkes Bay, New Zealand. The variety was determined to be distinct from the parent varieties by the following characteristics:

Packham's Triumph: ‘Nellie’ has medium weak tree vigour compared to medium vigour of ‘Packham's Triumph’, short pyriform shaped fruit that have 50-100% brown russet coverage compared to clear skinned fruit of ‘Packham's Triumph’.

President Heron: ‘Nellie’ has medium weak tree vigour compared to medium vigour of ‘President Heron’. The leaves of ‘Nellie’ are narrow and medium short with bluntly serrate leaf margin incisions compared to the leaves of ‘President Heron’ which are broad, medium length without leaf margin incisions. ‘Nellie’ has short pyriform fruit with 50-100% russet coverage and no skin blush colour while ‘President Heron’ has medium length pyriform fruit and some fruit have a red skin blush colour.

The new variety has been named ‘Nellie’. True to type budding and grafting onto Quince rootstock ‘BA29 with ‘Buerre Hardy’ pear as an interstock has been used to obtain plants. Asexual propagation by budding and grafting has shown that the unique combination of characteristics is consistently transferred through successive propagation.

### BRIEF DESCRIPTION OF THE DRAWINGS

The accompanying photographs show typical specimens of the tree, foliage, fruit of the new variety as depicted in colours nearly as true as is reasonably possible to make the same colour illustration of this character.

FIG. 1: side view of typical fruit of the pear variety ‘Nellie’.

FIG. 2: calyx end view of typical fruit of the pear variety ‘Nellie’.

FIG. 3: stem end view of a typical fruit of the pear variety ‘Nellie’

FIG. 4: cross sectional view of a typical fruit of the pear variety ‘Nellie’

FIG. 5: typical mature leaves of ‘Nellie’ showing the upper and lower surfaces.

FIG. 6: typical mature leaves of ‘Packhams Triumph’ left, ‘Nellie’ centre and ‘President Heron’ (right).

FIG. 7: side fruit view of ‘Nellie’ left and ‘Packhams Triumph’, (right).

FIG. 8: side fruit view of ‘Nellie’ left and ‘President Heron’ (right)

FIG. 9: fruit spur ‘Nellie’ left and ‘President Heron’ (right).

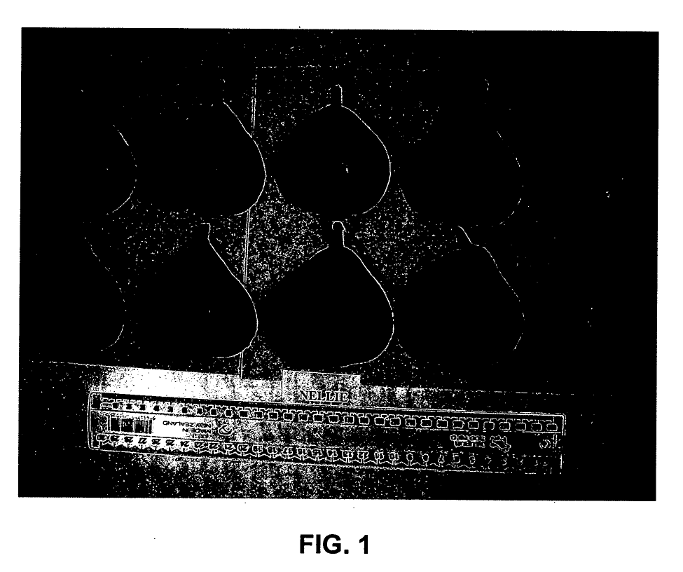

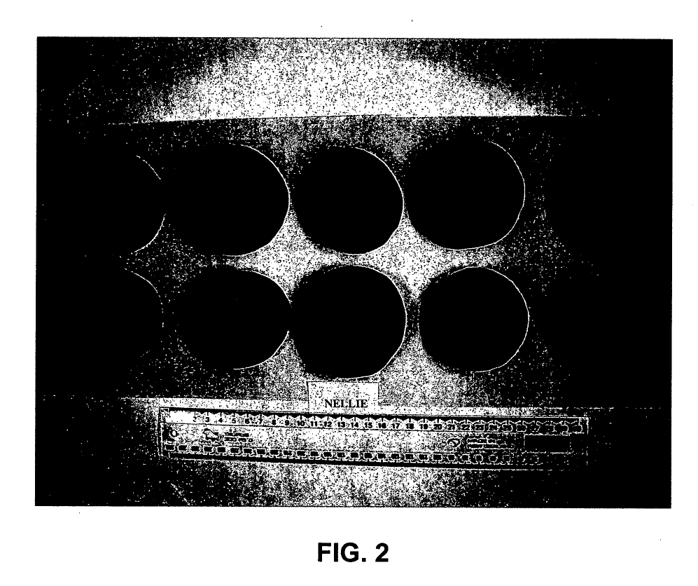

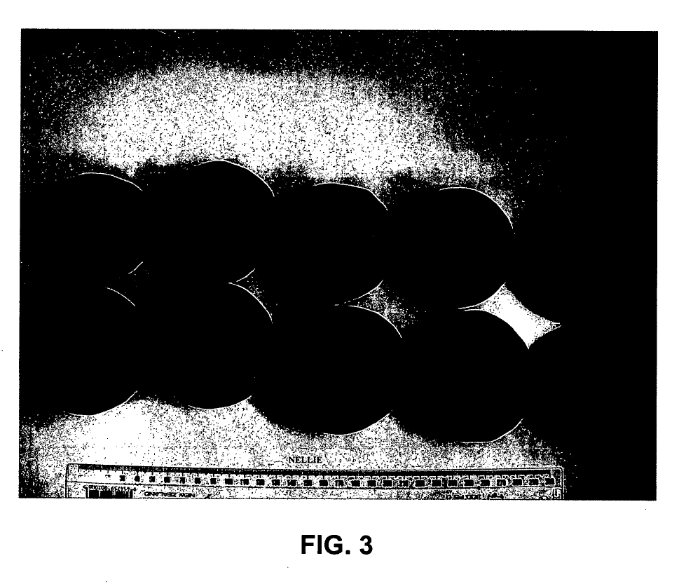

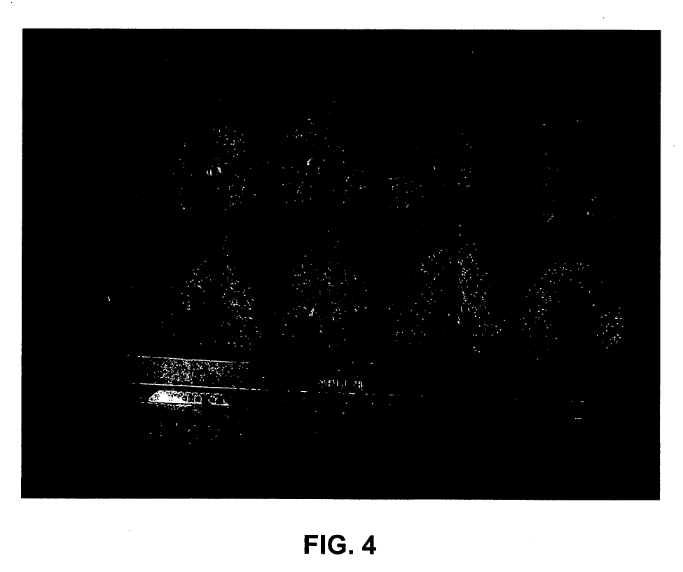

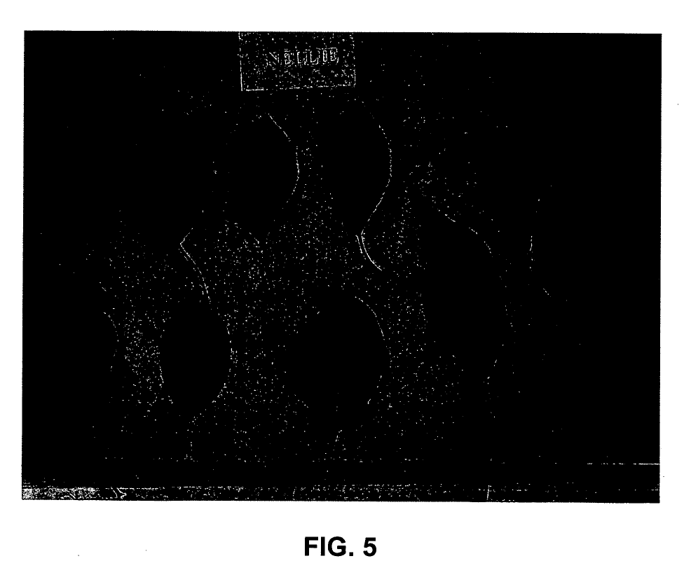

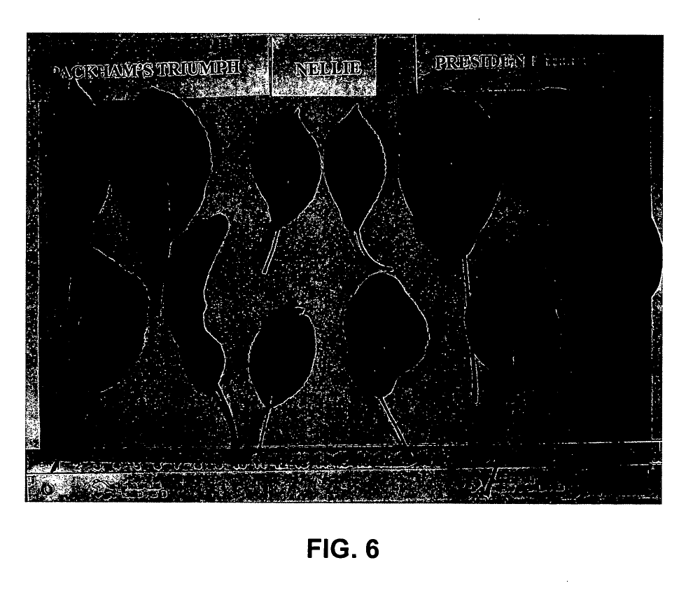

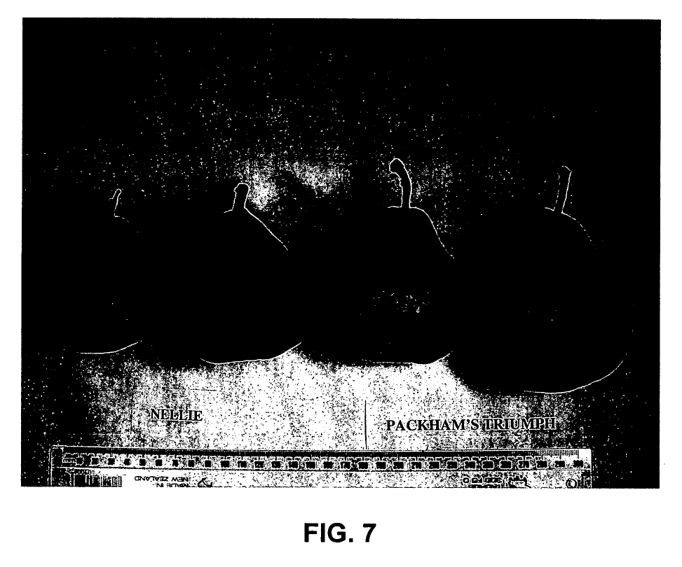

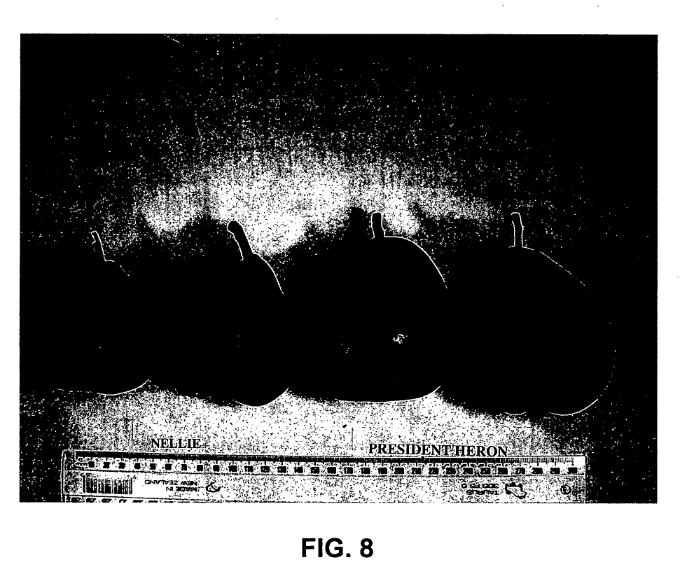

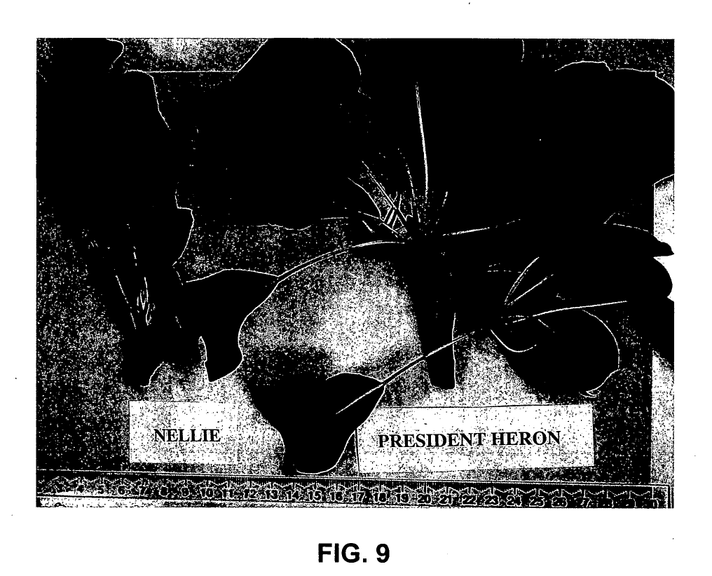

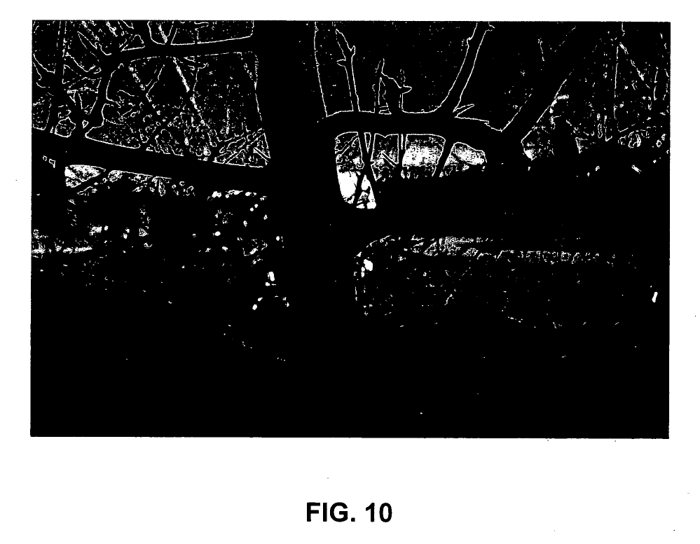

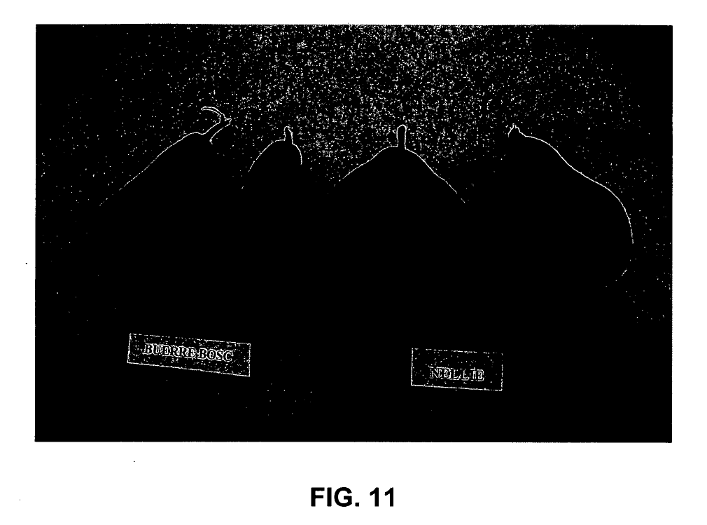

### MORPHOLOGICAL DESCRIPTION OF THE VARIETY

The following is a detailed description of the new variety with colour terminology in accordance with The Royal Horticultural Society Colour Chart (R.H.S.C.C.) 2001 edition.

The observations were made in the 2003-2004 season on trees grafted onto Quince ‘BA29’ rootstock with a ‘Buerre Hardy’ pear interstock, planted in 1999.
Tree: Vigour medium weak when grafted onto Quince ‘BA29’ rootstock with a ‘Buerre Hardy’ interstock; tree size medium, branching medium weak; habit semi upright; bearing on spurs.
Trunk: Bark is grey R.H.S.C.C. N200D when mature; diameter of 183 mm at a height of 200 mm.
Branches: Weak; number high; approximately 4 spurs per 100 mm of growth on two year old wood; annual growth numbers medium with a length of 300-500 mm; annual growth wood is greyed orange R.H.S.C.C. 165A.
Lenticels: Number on current years growth medium; size small.
Leaves: Length 63 mm; width 35 mm; average length/width ratio 1.82; downward pose, shape in cross section flat or upfolded, bluntly serrate indentations of the margin, shallow depth; shape of leaf blade apex (excluding pointed tip) acute; petiole average length 30.9 mm; stipules absent; colour of leaf blade green R.H.S.C.C. 137B.
Flower buds: Flower bud length medium.
Flowers: Bloom period, mid season, from 18 September to 9 October in Nelson, New Zealand, full bloom date 1 October; sepal length medium long; sepal attitude in relation to the corolla recurved; margins of petals apart; position of stigma in relation to stamens above; size of petal medium; shape of petal (excluding claw) ovate; shape of petal base (excluding claw) cuneate; length of claw petal medium long.
Fruit: Fruit is mature for consumption in the last week of February; harvest period is from 15 February to 25 February in Nelson, New Zealand.
Size: Medium; average fruit weight 203.6 g; average width 73 mm, average height 79 mm; fruit length/diameter ratio medium; maximum diameter slightly towards calyx of fruit.
Shape: Short pyriform; slightly asymmetric in longitudinal section; profile of the sides convex.
Eye basin: Average width 31 mm; average depth 4.4 mm, relative area of russet around eye basin large.
Stalk: Thickness medium, length medium, curvature of stalk absent or very weak, attitude of the stalk in relation to the axis of the fruit oblique; depth of stalk cavity very shallow or absent, average 1.7 mm, russet around the stem cavity large.
Sepal: Attitude of sepals erect.
Skin: Smooth; greasiness absent, cracking tendency of skin absent; background colour, yellow green R.H.S.C.C. 1C, lenticels small; russet coverage 50-100%, russet colour brown, over colour absent.
Overcolour: Absent.
Texture: Flesh texture medium; firmness soft; flesh juiciness medium, texture melting after cool storage and 7 days ambient period at 20° C.
Flavour: Sweetness medium; acidity low; honey, nutty, aniseed flavour with mild aroma.
Quality: Very good
Seeds: Five locules, 1 to 2 seeds per locule; medium seed size 9 mm long, 5.6 mm wide; colour black R.H.S.S.C.C. 202A.
Use: Dessert.
Production: Early mid season, regular cropping when thinned.
Management: Thinning of crop load in early summer is recommended.
Pest and disease: High level of field resistance to pear scab (Venturia pirina Adherh).

I claim:

1. A new and distinct variety of pear tree named ‘Nellie’ substantially as herein illustrated and described.
